(19) United States
(12) Patent Application Publication
(10) Pub. No.: US 2023/0105837 A1
(43) Pub. Date: Apr. 6, 2023

(54) ULTRASOUND DIAGNOSTIC APPARATUS, METHOD FOR CONTROLLING ULTRASOUND DIAGNOSTIC APPARATUS, AND NON-TRANSITORY COMPUTER-READABLE RECORDING MEDIUM STORING PROGRAM FOR CONTROLLING ULTRASOUND DIAGNOSTIC APPARATUS

(71) Applicant: Konica Minolta, Inc., Tokyo (JP)

(72) Inventors: Makiko URABE, Kanagawa (JP); Akihiro Kawabata, Tokyo (JP); Yoshihiro Takeda, Tokyo (JP)

(21) Appl. No.: 17/932,106

(22) Filed: Sep. 14, 2022

(30) Foreign Application Priority Data
Oct. 4, 2021 (JP) 2021-163468

(51) Int. Cl. A61B 8/08

(52) U.S. Cl. CPC A61B 8/5223; A61B 8/0891; A61B 8/13

(57) ABSTRACT

An ultrasound diagnostic apparatus according to the present disclosure includes a hardware processor that detects a region of a blood vessel appearing in a tomographic image; calculates a diameter of the blood vessel by analyzing the tomographic image; specifies, based on the diameter of the blood vessel calculated in each of a plurality of frames obtained within a predetermined period of the tomographic image, a frame of interest from among the plurality of frames, the frame of interest corresponding to a frame obtained when the diameter of the blood vessel is maximum and/or minimum; performs measurement on the blood vessel appearing in the frame of interest of the tomographic image, the measurement being measurement of a blood flow of a subject or a property of the blood vessel of the subject; and displays the frame of interest of the tomographic image at a predetermined timing.

### CROSS REFERENCE TO RELATED APPLICATIONS

The entire disclosure of Japanese Patent Application No. 2021-163468 filed on Oct. 4, 2021, is incorporated herein by reference in its entirety.

### BACKGROUND

### Technological Field

The present disclosure relates to an ultrasound diagnostic apparatus, a method for controlling the ultrasound diagnostic apparatus, and a non-transitory computer-readable recording medium storing a program for controlling the ultrasound diagnostic apparatus.

### Description of Related Art

There is known an ultrasound diagnostic apparatus that transmits ultrasound toward a subject, receives a reflected wave of the ultrasound, and performs predetermined signal processing on a reception signal, thereby visualizing, as a tomographic image, the shape, property, or behavior of an object inside the subject (see, for example, Japanese Patent Application Laid-Open No. 2008-168016).

In medical practice, a tomographic image of a blood vessel captured by the ultrasound diagnostic apparatus is used to measure the property of the blood vessel and the flow volume of blood flowing through the blood vessel, and diagnose the condition of the subject from a result of the measurement. For example, regarding ischemic diseases such as cerebral infarction and myocardial infarction, it is known that arteriosclerosis or stenosis occurs as a sign. To determine the development of arteriosclerosis or stenosis, a tomographic image of a blood vessel captured by the ultrasound diagnostic apparatus is used to measure, for example, an intima-media thickness (IMT) in a carotid artery and a flow volume (FV) in the carotid artery. In addition, measurement results of the IMT and FV of an artery other than a carotid artery are often used to diagnose the condition of the subject.

Accurate measurement of the IMT and FV may enable accurate evaluation of a sign such as the development of arteriosclerosis or stenosis. However, the diameter of a blood vessel, such as a carotid artery, changes in accordance with beating of an internal organ or the heart. Thus, it is prescribed in medical practice that IMT measurement or FV measurement is to be typically performed by using a tomographic image captured when the diameter of the blood vessel is maximum or a tomographic image captured when the diameter of the blood vessel is minimum, in order to fix an evaluation criterion. For example, measurement at end-diastole is recommended in IMT measurement. Because the diameter of a blood vessel becomes close to the minimum at end-diastole, IMT measurement is often performed in the site by using a tomographic image captured when the diameter of a blood vessel is minimum.

FIG. 17 is a diagram illustrating an example of a tomographic image of a blood vessel (a carotid artery herein) captured by an ultrasound diagnostic apparatus. In this tomographic image, the blood vessel extends in a lateral direction, and the diameter of the blood vessel is calculated as, for example, the distance between an upper vessel wall and a lower vessel wall with an intravascular lumen therebetween (width D1 in FIG. 17).

To perform measurement of the IMT or FV in a blood vessel, such as a carotid artery (hereinafter referred to as blood vessel measurement) by using an ultrasound diagnostic apparatus according to the related art, a user needs to perform an operation of scanning the blood vessel for a certain time (for example, several minutes) by using an ultrasound probe, searching a time-series frame group captured as a moving image (for example, frame data for several past minutes) for a frame in which the blood vessel diameter is maximum or minimum by performing a cine operation (an operation of visually checking each frame of a tomographic image), designating the frame as a result of the search, and specifying the position of the blood vessel as a measurement target.

Such an operation is very complicated for the user and is a factor of taking time in blood vessel measurement. In addition, such a measurement method depends on intuition of the user, and there is room for improvement in terms of measurement accuracy and reliability.

### SUMMARY

The present disclosure has been made in view of the above issues, and objects of the present disclosure are to provide an ultrasound diagnostic apparatus, a method for controlling the ultrasound diagnostic apparatus, and a non-transitory computer-readable recording medium storing a program for controlling the ultrasound diagnostic apparatus that are capable of, at the time of performing blood vessel measurement, reducing the work load of a user and increasing the accuracy and reliability of the blood vessel measurement.

To achieve at least one of the abovementioned objects, according to an aspect of the present invention, an ultrasound diagnostic apparatus reflecting one aspect of the present invention generates a tomographic image of a subject by transmitting and receiving ultrasound, and comprises a hardware processor that:

detects a region of a blood vessel appearing in the tomographic image;

calculates a diameter of the blood vessel by analyzing the tomographic image;

specifies, based on the diameter of the blood vessel calculated in each of a plurality of frames obtained within a predetermined period of the tomographic image, a frame of interest from among the plurality of frames, the frame of interest corresponding to a frame obtained when the diameter of the blood vessel is maximum and/or minimum;

performs measurement on the blood vessel appearing in the frame of interest of the tomographic image, the measurement being measurement of a blood flow of the subject or a property of the blood vessel of the subject; and

displays the frame of interest of the tomographic image at a predetermined timing.

To achieve at least one of the abovementioned objects, according to an aspect of the present invention, a method reflecting one aspect of the present invention is a method for controlling an ultrasound diagnostic apparatus that generates a tomographic image of a subject by transmitting and receiving ultrasound. The method comprises:

detecting a region of a blood vessel appearing in the tomographic image;

calculating a diameter of the blood vessel by analyzing the tomographic image;

specifying, based on the diameter of the blood vessel calculated in each of a plurality of frames obtained within a predetermined period of the tomographic image, a frame of interest from among the plurality of frames, the frame of interest corresponding to a frame obtained when the diameter of the blood vessel is maximum and/or minimum;

performing measurement on the blood vessel appearing in the frame of interest of the tomographic image, the measurement being measurement of a blood flow of the subject or a property of the blood vessel of the subject; and

displaying the frame of interest of the tomographic image at a predetermined timing.

To achieve at least one of the abovementioned objects, according to an aspect of the present invention, a non-transitory computer-readable recording medium reflecting one aspect of the present invention is a non-transitory computer-readable recording medium storing a program for controlling an ultrasound diagnostic apparatus that generates a tomographic image of a subject by transmitting and receiving ultrasound. The program causes a computer to perform:

detecting a region of a blood vessel appearing in the tomographic image;

calculating a diameter of the blood vessel by analyzing the tomographic image;

specifying, based on the diameter of the blood vessel calculated in each of a plurality of frames obtained within a predetermined period of the tomographic image, a frame of interest from among the plurality of frames, the frame of interest corresponding to a frame obtained when the diameter of the blood vessel is maximum and/or minimum;

performing measurement on the blood vessel appearing in the frame of interest of the tomographic image, the measurement being measurement of a blood flow of the subject or a property of the blood vessel of the subject; and

displaying the frame of interest of the tomographic image at a predetermined timing.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a diagram illustrating an example of the external appearance of an ultrasound diagnostic apparatus according to one embodiment of the present disclosure;

FIG. 2 is a diagram illustrating an example of the overall configuration of an ultrasound diagnostic apparatus according to one embodiment of the present disclosure;

FIG. 3 is a diagram illustrating an example of a display image generated by a display processing section when a scanning operation is executed in an ultrasound diagnostic apparatus according to one embodiment of the present disclosure;

FIG. 4 is a diagram illustrating an example of a display image generated by a display processing section when an ultrasound diagnostic apparatus according to one embodiment of the present disclosure is executing measurement after stopping a scanning operation;

FIG. 5 is a diagram illustrating an example configuration of a blood vessel measurement support section according to one embodiment of the present disclosure;

FIG. 6 is a flowchart illustrating an example of a process executed by a blood vessel detecting section according to one embodiment of the present disclosure;

FIG. 7 is a diagram for schematically describing an example of a process executed by a blood vessel detecting section according to one embodiment of the present disclosure;

FIG. 8 is a diagram for schematically describing an example of a process of calculating the diameter of a blood vessel by a blood vessel diameter calculating section according to one embodiment of the present disclosure;

FIG. 9 is a diagram for describing an IMT measurement process performed by a measuring section according to one embodiment of the present disclosure;

FIG. 10 is a flowchart illustrating an example of operation of a control device according to one embodiment of the present disclosure;

FIG. 11 is a flowchart illustrating an example of operation of a control device of an ultrasound diagnostic apparatus according to a first modification;

FIG. 12 is a diagram illustrating an example of an image showing a temporal change in the diameter of a blood vessel displayed by an ultrasound diagnostic apparatus according to a second modification;

FIG. 13 is a diagram illustrating an example of display of a cine bar in an ultrasound diagnostic apparatus according to a third modification;

FIG. 14 is a diagram illustrating an example of candidate frame display displayed by an ultrasound diagnostic apparatus according to a fourth modification;

FIG. 15 is a diagram illustrating an example of a user interface image that is caused to be displayed by a setting section of an ultrasound diagnostic apparatus according to a fifth modification;

FIG. 16 is a diagram illustrating an example of a user interface image that is caused to be displayed by a setting section of an ultrasound diagnostic apparatus according to the fifth modification; and

FIG. 17 is a diagram illustrating an example of a tomographic image of a blood vessel (a carotid artery herein) captured by an ultrasound diagnostic apparatus.

### DETAILED DESCRIPTION OF EMBODIMENTS

Hereinafter, one or more embodiments of the present invention will be described with reference to the drawings. However, the scope of the invention is not limited to the disclosed embodiments.

In the specification and drawings, elements having substantially the same functions are denoted by the same reference numerals, and a repeated description will be omitted.

### Configuration of Ultrasound Diagnostic Apparatus

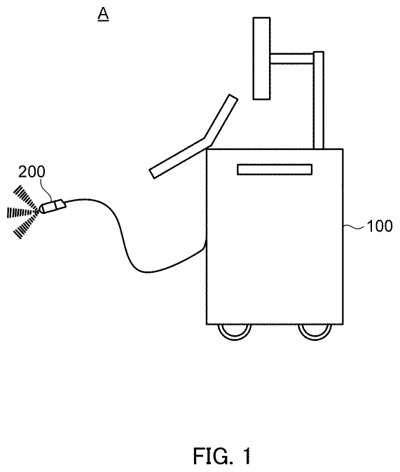

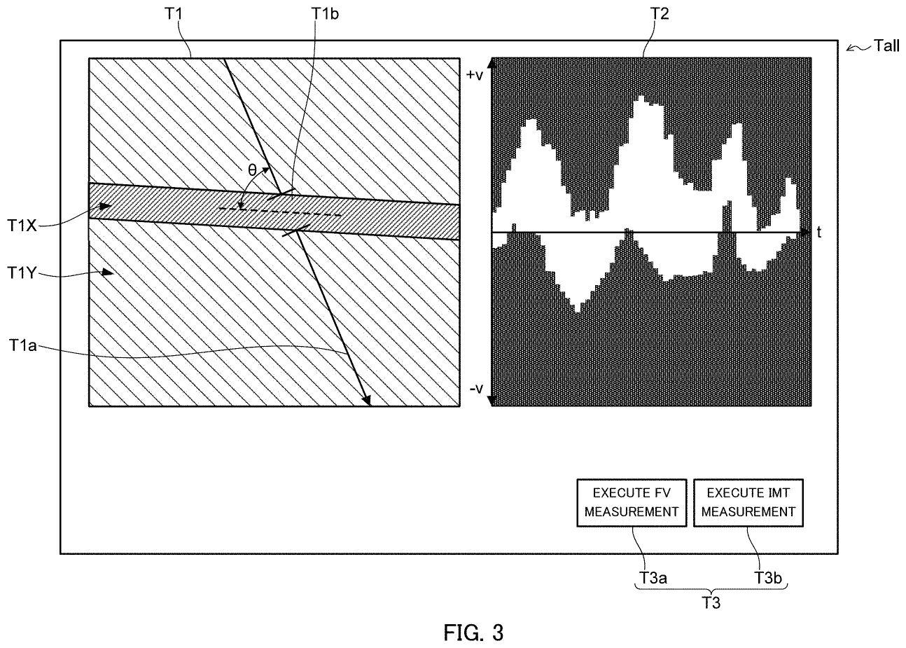

Hereinafter, the configuration of an ultrasound diagnostic apparatus according to one embodiment of the present disclosure (hereinafter referred to as “ultrasound diagnostic apparatus A”) will be described with reference to FIGS. 1 to 3. In the present embodiment, a description will be given of a case in which ultrasound diagnostic apparatus A performs a brightness (B)-mode operation and a pulsed-wave (PW)-Doppler-mode operation in a time division manner and generates a tomographic image and a Doppler spectrum image (see FIG. 3). Note that ultrasound diagnostic apparatus A according to one embodiment of the present disclosure may implement a color Doppler mode or a power Doppler mode instead of the PW Doppler mode.

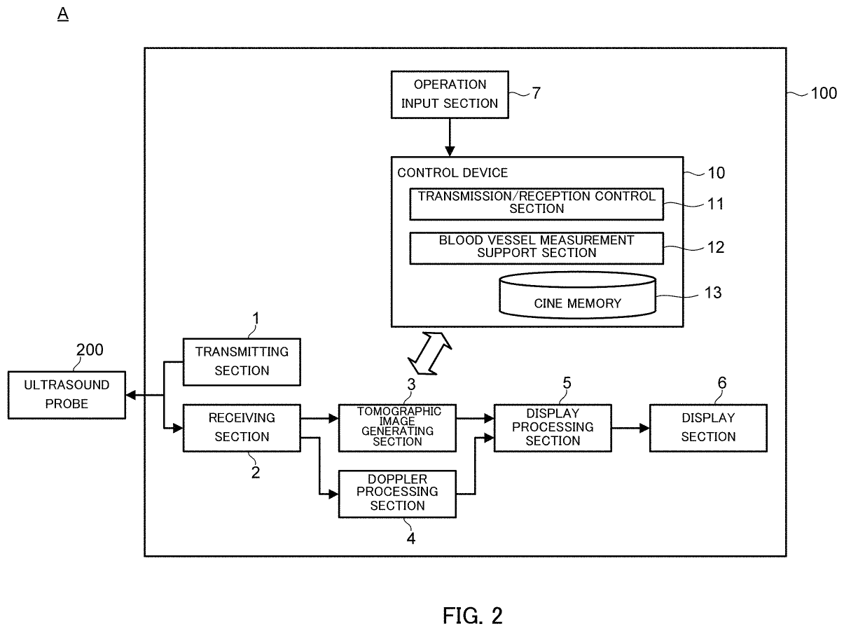

FIG. 1 is a diagram illustrating an example of the external appearance of ultrasound diagnostic apparatus A. FIG. 2 is a diagram illustrating an example of the overall configuration of ultrasound diagnostic apparatus A.

Ultrasound diagnostic apparatus A is used to visualize the shape, property, or behavior of an object inside a subject as an ultrasound image and perform image diagnosis. Ultrasound diagnostic apparatus A includes ultrasound diagnostic apparatus body 100 and ultrasound probe 200.

Ultrasound probe 200 functions as an acoustic sensor that transmits an ultrasound beam (about 1 to 30 MHz herein) into a subject (for example, a human body), receives an ultrasound echo of the transmitted ultrasound beam reflected in the subject, and converts the ultrasound echo into an electric signal.

A user operates ultrasound diagnostic apparatus A with an ultrasound beam transmission/reception surface of ultrasound probe 200 being in contact with the subject, and performs ultrasound diagnosis. Ultrasound probe 200 herein transmits an ultrasound beam into the subject through an outer surface of the subject, and receives an ultrasound echo of the ultrasound beam. Alternatively, an ultrasound probe to be inserted into an alimentary canal, a blood vessel, or a body cavity may be used as ultrasound probe 200. In addition, any type of ultrasound probe, such as a convex probe, a linear probe, a sector probe, or a three-dimensional probe, may be used as ultrasound probe 200.

Ultrasound probe 200 includes, for example, a plurality of transducers (for example, piezoelectric elements) arranged in matrix, and a channel switching section (for example, a multiplexer) for controlling ON/OFF switching of the driving states of the plurality of transducers individually or in units of blocks (hereinafter referred to as “channels”).

Each transducer of ultrasound probe 200 converts a voltage pulse generated by ultrasound diagnostic apparatus body 100 (transmitting section 1) into an ultrasound beam, transmits the ultrasound beam into the subject, receives an ultrasound echo reflected in the subject, converts the ultrasound echo into an electric signal (hereinafter referred to as “reception signal”), and outputs the reception signal to ultrasound diagnostic apparatus body 100 (receiving section 2).

Ultrasound diagnostic apparatus body 100 includes transmitting section 1, receiving section 2, tomographic image generating section 3, Doppler processing section 4, display processing section 5, display section 6, operation input section 7, and control device 10.

Transmitting section 1 is a transmitter that transmits a voltage pulse serving as a driving signal to ultrasound probe 200. Transmitting section 1 includes, for example, a high-frequency pulse oscillator, a pulse setting section, and so forth. Transmitting section 1 regulates a voltage pulse generated by the high-frequency pulse oscillator so that the voltage pulse has a voltage amplitude, a pulse width, and a transmission timing set by the pulse setting section, and transmits the voltage pulse for each of channels of ultrasound probe 200.

Transmitting section 1 includes pulse setting sections each for a corresponding one of a plurality of channels of ultrasound probe 200, and is capable of setting the voltage amplitude, pulse width, and transmission timing of a voltage pulse for each of the plurality of channels. For example, transmitting section 1 sets an appropriate delay time for each of the plurality of channels, thereby changing a target depth or generating a different pulse waveform (for example, transmits a 1-wave pulse in the B mode, and transmits 4-wave pulse in the PW Doppler mode).

Receiving section 2 is a receiver that performs reception processing on a reception signal related to an ultrasound echo generated by ultrasound probe 200. Receiving section 2 includes a preamplifier, an analog-to-digital (AD) converter, a reception beam former, and a processing system switching section.

In receiving section 2, the preamplifier amplifies a reception signal related to a weak ultrasound echo for each channel, and the AD converter converts the reception signal into a digital signal. In addition, in receiving section 2, the reception beam former performs phasing addition on reception signals of the respective channels to combine the reception signals into one signal, which serves as acoustic line data. In addition, in receiving section 2, the processing system switching section switches and controls the destination of a reception signal generated by the reception beam former, and outputs the reception signal to tomographic image generating section 3 or Doppler processing section 4 in accordance with an operation mode to be executed.

Tomographic image generating section 3 obtains a reception signal from the receiving section 2 during a B-mode operation and generates a tomographic image (also referred to as a B-mode image) of the inside of the subject.

For example, when ultrasound probe 200 has transmitted a pulsed ultrasound beam in a depth direction, tomographic image generating section 3 temporally continuously stores the signal intensity of an ultrasound echo detected thereafter in a line memory. As the inside of the subject is scanned with the ultrasound beam from ultrasound probe 200, tomographic image generating section 3 sequentially stores the signal intensities of ultrasound echoes at individual scanning positions in the line memory, and generates two-dimensional data in units of frames. Subsequently, tomographic image generating section 3 converts the signal intensities of ultrasound echoes detected at individual positions in the subject into luminance values, thereby generating a tomographic image.

Tomographic image generating section 3 includes, for example, an envelope detector circuit, a dynamic filter, and a logarithmic compression circuit. The envelope detector circuit detects the envelope of a reception signal and detects a signal intensity. The logarithmic compression circuit performs logarithmic compression on the signal intensity of the reception signal detected by the envelope detector circuit. The dynamic filter is a band-pass filter having a frequency characteristic changed in accordance with a depth, and removes a noise component included in the reception signal.

Doppler processing section 4 obtains a reception signal from receiving section 2 and detects a Doppler shift frequency for the transmission frequency of an ultrasound echo from a blood flow, during a PW-Doppler-mode operation, a color-Doppler-mode operation, or a power-Doppler-mode operation. Doppler processing section 4 selectively extracts an ultrasound echo from a sample gate position or a region of interest (ROI) set by input of a user operation or by an automatic blood vessel detection function, thereby detecting an ultrasound echo from a blood flow in the subject and a Doppler shift frequency for the transmission frequency.

For example, in a PW-Doppler-mode operation, while ultrasound probe 200 is transmitting a pulsed ultrasound beam at a regular interval in accordance with a pulse repetition frequency, Doppler processing section 4 samples a reception signal related to an ultrasound echo in synchronization with the pulse repetition frequency. Subsequently, for example, Doppler processing section 4 detects a Doppler shift frequency, based on a phase difference between an ultrasound echo related to the n-th ultrasound beam from a sample gate position and an ultrasound echo related to the n+1-th ultrasound beam from the same sample gate position.

Doppler processing section 4 includes, for example, an orthogonal detector section, a low pass filter, a range gate, and a fast Fourier transform (FFT) analysis section. The orthogonal detector section mixes a reception signal with a reference signal having the same phase as that of a transmitted ultrasound beam and a reference signal having a phase different from that of the transmitted ultrasound beam by π/2, thereby generating an orthogonal detection signal. The low pass filter removes a high-frequency component of the orthogonal detection signal, thereby generating a reception signal related to a Doppler shift frequency. The range gate obtains only an ultrasound echo from a sample gate position. The FFT analysis section calculates the Doppler shift frequency of an ultrasound echo, based on a temporal change in the reception signal output from the range gate.

Display processing section 5 generates a display image to be displayed on display section 6 under control by control device 10.

FIG. 3 is a diagram illustrating an example of a display image generated by display processing section 5 in ultrasound diagnostic apparatus A when a scanning operation is executed (when a B-mode operation and a PW-Doppler-mode operation are executed in parallel herein). Hereinafter, such a display image will be referred to as a “scanning-operation display screen”.

In FIG. 3, Tall denotes the entire region of the display image, T1 denotes a tomographic image, T2 denotes a Doppler spectrum image, and T3 denotes an icon group for inputting an instruction to start measurement of a blood vessel by a user. In tomographic image T1, T1X denotes a blood flow region, T1Y denotes a tissue region, T1a denotes a steering angle of an ultrasound beam in a PW-Doppler-mode operation, and T1b denotes a sample gate position of an ultrasound beam in the PW-Doppler-mode operation. In icon group T3, T3a denotes an icon for inputting an instruction to start FV measurement, and T3b denotes an icon for inputting an instruction to start IMT measurement.

For example, at the time of executing a scanning operation in a B-mode operation or a Doppler-mode operation (PW-Doppler-mode operation herein), display processing section 5 obtains a tomographic image output from tomographic image generating section 3, obtains a Doppler shift frequency output from Doppler processing section 4, and generates the display image as illustrated in FIG. 3 from the tomographic image and the Doppler shift frequency. When the tomographic image generated by tomographic image generating section 3 is successively updated, display processing section 5 successively updates tomographic image T1 displayed in the display image accordingly. When the Doppler shift frequency of an ultrasound echo calculated by Doppler processing section 4 is successively updated, display processing section 5 successively updates Doppler spectrum image T2 displayed in the display image accordingly.

Doppler spectrum image T2 is an image depicting a time-series distribution of blood flow velocity, in which the horizontal axis represents time and the vertical axis represents blood flow velocity. In a Doppler spectrum image, for example, blood flow velocities at individual time points are expressed by one line or the like, and powers at individual blood flow velocities (i.e., individual frequencies) are expressed by the luminances of pixels (in FIG. 3, the illustration of changes in luminance is omitted). The blood flow velocity for depicting Doppler spectrum image T2 is calculated from a Doppler shift frequency by using, for example, the following equation (1) in consideration with an angle correction value for a crossing angle formed by an ultrasound beam direction and a blood flow direction.

V=c/2 cos θ×Fd/F0 (1)

(Note that V represents blood flow velocity, F0 represents transmission frequency of ultrasound beam, Fd represents Doppler shift frequency, c represents acoustic velocity in living body, and θ represents angle correction value.)

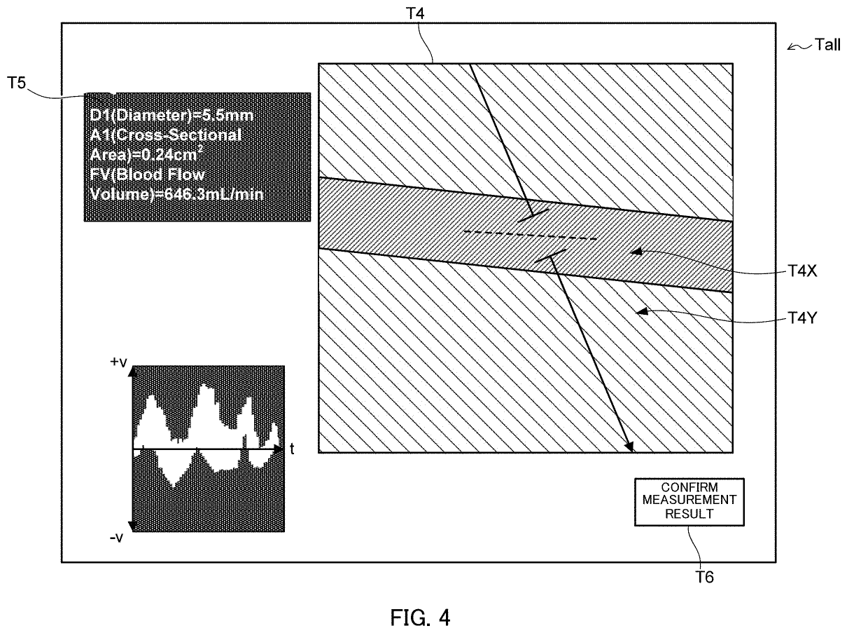

FIG. 4 is a diagram illustrating an example of a display image generated by display processing section 5 when ultrasound diagnostic apparatus A is executing measurement after stopping a scanning operation. Hereinafter, such a display image will be referred to as a “measurement display screen”.

In FIG. 4, Tall denotes the entire region of the display image, T4 denotes a tomographic image referred to during measurement (herein a tomographic image obtained when the diameter of the blood vessel is maximum), T4X denotes a blood flow region, T4Y denotes a tissue region, T5 denotes an image showing a measurement result, and T6 denotes an icon for inputting an instruction to confirm a measurement result by a user. In FIG. 4, measurement result T5 indicates the diameter and cross-sectional area of the blood vessel in the tomographic image referred to during the measurement, and the blood flow volume in the blood vessel.

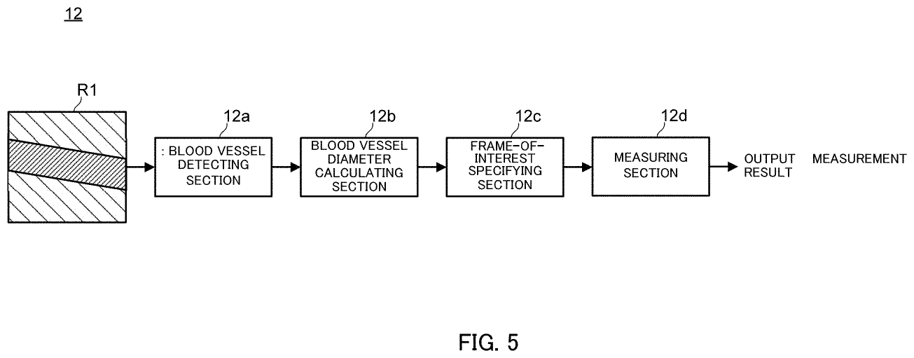

For example, when the user operates icon T3a or T3b for inputting an instruction to start measurement of the blood vessel, ultrasound diagnostic apparatus A stops a scanning operation and shift to a measurement execution mode. The measurement execution mode is a mode in which blood vessel measurement support section 12 of control device 10 (described below) performs arithmetic processing, and is a mode in which a tomographic image suitable as a measurement target is automatically specified from among frames obtained through the scanning operation performed so far, and measurement of the blood flow of a subject or the property of the blood vessel of the subject (for example, FV measurement or IMT measurement) is performed using the tomographic image (described below with reference to FIGS. 5 to 10).

At this time, display processing section 5 performs a screen shift from the scanning-operation display image (FIG. 3) (i.e., a first screen mode of automatically updating a continuously generated tomographic image) to the measurement display image (FIG. 4) (i.e., a second screen mode of displaying a still image of a frame of interest of the tomographic image specified by frame-of-interest specifying section 12c described below), and provides the user with tomographic image T4 (still image) as a measurement target and measurement result T5.

Tomographic image generating section 3, Doppler processing section 4, and display processing section 5 are implemented by, for example, a digital processing circuit constituted by a digital signal processor (DSP) or the like. The configurations of these sections can be variously modified, for example, a part or the entirety thereof may be implemented by a hardware circuit or may be implemented by arithmetic processing executed in accordance with a program.

Display section 6 is a display that displays a display image generated by display processing section 5 and is constituted by, for example, a liquid crystal display.

Operation input section 7 is a user interface used by a user to perform an input operation, and is constituted by, for example, a press button switch, a keyboard, a mouse, and so forth. Operation input section 7 converts an operation performed by the user into an operation signal, and inputs the operation signal to control device 10.

Control device 10 transmits signals to and receives signals from ultrasound probe 200, transmitting section 1, receiving section 2, tomographic image generating section 3, Doppler processing section 4, display processing section 5, display section 6, and operation input section 7, and centrally controls these sections. Control device 10 includes, for example, a central processing unit (CPU), a read only memory (ROM), a random access memory (RAM), and so forth. The functions of control device 10 are implemented by the CPU referring to a control program and various data stored in the ROM and the RAM.

Control device 10 includes cine memory 13 that temporarily stores a tomographic image (frame data) of the latest several minutes of a tomographic image continuously generated by tomographic image generating section 3 such that the tomographic image can be reproduced as a moving image. Cine memory 13 is typically configured to store a tomographic image of a certain time period as time-series data and sequentially erase old data when the tomographic image stored therein exceeds the storage capacity.

Control device 10 includes transmission/reception control section 11 and blood vessel measurement support section 12.

Transmission/reception control section 11 controls the channel switching section (not illustrated) of ultrasound probe 200 and selects a channel as a driving target from among a plurality of channels. Transmission/reception control section 11 controls each of transmitting section 1 and receiving section 2 to transmit and receive ultrasound to/from the channel as the driving target.

At the time of a B-mode operation (i.e., at the time of generating a tomographic image), transmission/reception control section 11 sequentially drives channels as driving targets among a plurality of channels in a scanning direction, thereby causing ultrasound probe 200 to perform ultrasound scanning in a subject.

At the time of a PW-Doppler-mode operation, a color-Doppler-mode operation, or a power-Doppler-mode operation (i.e., at the time of measuring a blood flow velocity), transmission/reception control section 11 selectively drives a plurality of transducers provided in ultrasound probe 200 so that an ultrasound beam is transmitted from ultrasound probe 200 to a sample gate position or ROI in a subject at a predetermined angle. At this time, transmission/reception control section 11 controls transmitting section 1 so as to repeatedly transmit a pulsed ultrasound beam (burst wave) from ultrasound probe 200 at a predetermined pulse repetition frequency, and also controls receiving section 2 so as to receive an ultrasound echo of the ultrasound beam.

Transmission/reception control section 11 determines ultrasound beam transmission/reception conditions in accordance with the type of ultrasound probe 200 (for example, convex, sector, linear, or the like), the depth of a target to be imaged in a subject, the imaging mode (for example, B mode, PW Doppler mode, color Doppler mode, or power Doppler mode), and so forth set by a user via operation input section 7.

Blood vessel measurement support section 12 automatically specifies a tomographic image suitable as a measurement target from a frame group in a predetermined period obtained by a scanning operation, and executes measurement of the blood flow of the subject or the property of the blood vessel of the subject (for example, FV measurement, IMT measurement, or the like) by using the tomographic image.

### Detailed Configuration of Blood Vessel Measurement Support Section 12

Next, a detailed configuration of blood vessel measurement support section 12 will be described with reference to FIGS. 5 to 10.

FIG. 5 is a diagram illustrating an example configuration of blood vessel measurement support section 12.

Blood vessel measurement support section 12 includes blood vessel detecting section 12a, blood vessel diameter calculating section 12b, frame-of-interest specifying section 12c, and measuring section 12d.

### Blood Vessel Detecting Section 12a

Blood vessel detecting section 12a obtains tomographic image R1 generated by tomographic image generating section 3, and detects a blood vessel appearing in tomographic image R1 by using image information of tomographic image R1. Blood vessel detecting section 12a detects a blood vessel appearing in tomographic image R1 by known template matching by using, for example, data of blood vessel patterns (hereinafter also referred to as “blood vessel template image”) stored in advance in a memory (not illustrated).

Subsequently, blood vessel detecting section 12a outputs, for example, a region in which the blood vessel appears most clearly in tomographic image R1, as the position of the blood vessel for which the diameter is to be measured by blood vessel diameter calculating section 12b.

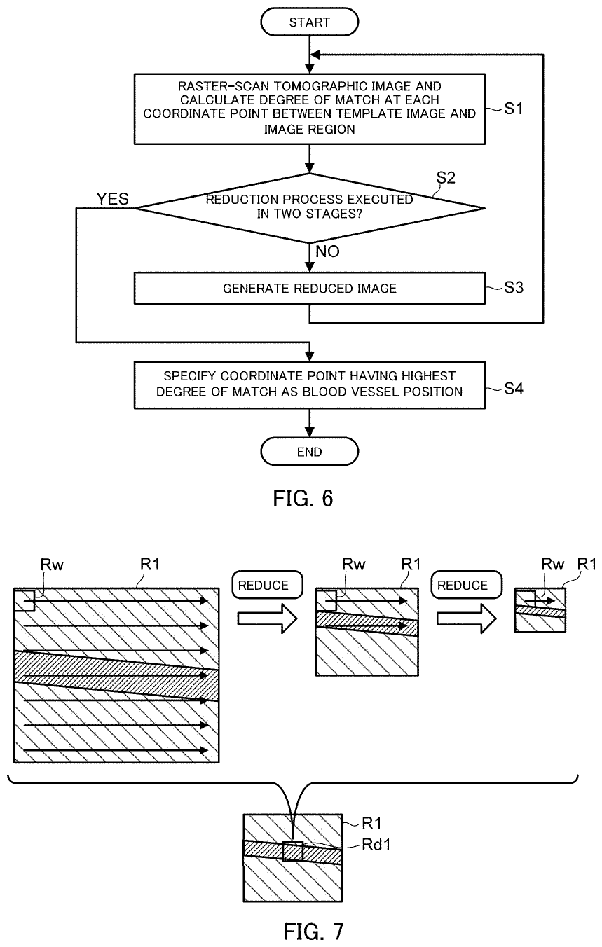

FIG. 6 is a flowchart illustrating an example of a process executed by blood vessel detecting section 12a. FIG. 7 is a diagram for schematically describing an example of a process executed by blood vessel detecting section 12a.

First, in step S1, blood vessel detecting section 12a reads out template image Rw of a blood vessel stored in the ROM or the like of control device 10. Subsequently, for example, blood vessel detecting section 12a sequentially sets, in tomographic image R1, image regions as targets to be compared (hereinafter referred to as “comparison target regions”) each having the same size as template image Rw (for example, 100 pixels×100 pixels) so as to raster-scan tomographic image R1, and calculates, for each comparison target region, the degree of match (i.e., the degree of similarity) with template image Rw. Subsequently, blood vessel detecting section 12a calculates, for each of coordinate points in tomographic image R1, the degree of match with temperate image Rw.

Template image Rw of a blood vessel referred to by blood vessel detecting section 12a may be, for example, an image in which a blood vessel region extends in a lateral direction in an image center region and is sandwiched between tissue regions in an up-down direction (i.e., a blood vessel longer-axis image).

Subsequently, in step S2, blood vessel detecting section 12a determines whether a reduction process in the following step S3 has been executed in two stages. If the reduction process in step S3 has been executed in two stages (YES in step S2), the process proceeds to step S4. If the reduction process in step S3 has not been executed in two stages (NO in step S2), the process proceeds to step S3.

Subsequently, in step S3, blood vessel detecting section 12a reduces tomographic image R1 by a predetermined magnitude (for example, 0.9 times) to generate a reduced image. Subsequently, the process returns to step S1, where blood vessel detecting section 12a performs template matching on the reduced image by using template image Rw of a blood vessel, and calculates the degree of match for each of coordinate points of the reduced image. At this time, the size of template image Rw of a blood vessel is not changed, and template image Rw of a blood vessel applied to original tomographic image R1 is used.

The search process using this reduced image is a process performed in consideration of a case where the size of the blood vessel appearing in tomographic image R1 is different from that in template image Rw.

Subsequently, in step S4, blood vessel detecting section 12a specifies a coordinate point having a highest degree of match from among the individual coordinate points of tomographic image R1, the individual coordinate points of the reduced image, and the individual coordinate points of the re-reduced image (tomographic image R1 reduced in two stages).

With the above-described process, blood vessel detecting section 12a searches for a region in which the blood vessel appears most clearly in tomographic image R1, and outputs the region (i.e., a center coordinate point) as the position of the blood vessel to be referred to in the following process. Specifically, the position of the blood vessel detected in this manner is also used as the position of the blood vessel as a target for which the diameter is to be measured by blood vessel diameter calculating section 12b, and a reference position for performing measurement by measuring section 12d.

The position of the blood vessel detected by blood vessel detecting section 12a may be used as a sample gate position at the time of executing the Doppler mode. In other words, to set a sample gate position at the time of executing the Doppler mode, the position of the blood vessel detected by blood vessel detecting section 12a may be used by blood vessel diameter calculating section 12b, frame-of-interest specifying section 12c, measuring section 12d, and so forth.

Blood vessel detecting section 12a may detect the position of the blood vessel in every frame of tomographic image R1 continuously generated by tomographic image generating section 3. Alternatively, blood vessel detecting section 12a may detect the position of the blood vessel in only a frame of tomographic image R1 obtained at an appropriate timing among frames of tomographic image R1 continuously generated by tomographic image generating section 3. In this case, for example, in a frame of tomographic image R1 in which detection of the position of the blood vessel has not been performed among frames of tomographic image R1 continuously generated by tomographic image generating section 3, the position of the blood vessel may be estimated based on the position of the blood vessel detected in a frame of tomographic image R1 in which detection of the position of the blood vessel has been performed.

Accordingly, every time tomographic image R1 is updated, the position of the blood vessel set by blood vessel detecting section 12a changes, and a situation can be prevented from occurring where appropriate comparison of the blood vessel diameter is not made in the process performed by frame-of-interest specifying section 12c described below.

The method of detecting a blood vessel by blood vessel detecting section 12a is not specified. A discriminator (for example, a convolutional neural network (CNN)) trained by machine learning or the like may be used instead of template matching.

### Blood Vessel Diameter Calculating Section 12b

Blood vessel diameter calculating section 12b obtains detected position information of the blood vessel from blood vessel detecting section 12a, and analyzes tomographic image R1 to calculate the diameter of blood vessel Rd1 present at the position detected by blood vessel detecting section 12a (hereinafter simply referred to as “blood vessel Rd1”).

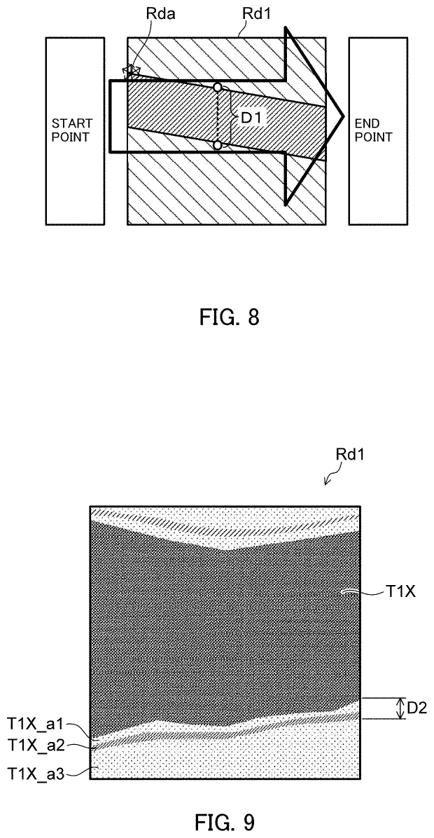

FIG. 8 is a diagram for schematically describing an example of a process of calculating the diameter of blood vessel Rd1 by blood vessel diameter calculating section 12b.

For example, blood vessel diameter calculating section 12b searches for a path while regarding a path having a strong and smoothly continued edge as a boundary between a blood vessel and a tissue outside the blood vessel in an image region at the detection position of blood vessel Rd1. Specifically, blood vessel diameter calculating section 12b replaces boundary detection with path search of searching for a path of minimizing the cost, and searches for a path of minimizing the cost from the left end (Rda in FIG. 8) of the image region at the detection position of blood vessel Rd1, with a direction in which the edge is small and a direction in which the path is not smooth being a direction of increasing the cost. Accordingly, blood vessel diameter calculating section 12b detects a boundary position between an upper side wall portion of blood vessel Rd1 and a tissue outside the blood vessel, and a boundary position between a lower side wall portion of blood vessel Rd1 and a tissue outside the blood vessel. Subsequently, blood vessel diameter calculating section 12b calculates, as the diameter of blood vessel Rd1 (represented by D1 in FIG. 8), the distance between the boundary position of the upper side wall portion of blood vessel Rd1 and the boundary position of the lower side wall portion of blood vessel Rd1 (for example, a maximum value of a blood vessel width calculated at individual positions in a lateral direction).

The diameter of blood vessel Rd1 is defined, in a simple manner, as the distance between the boundary position of the upper side wall portion of blood vessel Rd1 and the boundary position of the lower side wall portion of blood vessel Rd1 in the depth direction. To more accurately calculate the diameter of blood vessel Rd1, the diameter of blood vessel Rd1 may be defined as the distance between the boundary position of the upper side wall portion of blood vessel Rd1 and the boundary position of the lower side wall portion of blood vessel Rd1 in a direction orthogonal to the direction in which blood vessel Rd1 extends. In this case, for example, the direction in which blood vessel Rd1 extends may be calculated based on, for example, an average value of the direction in which the boundary of the upper side wall portion of blood vessel Rd1 extends and the direction in which the boundary of the lower side wall portion of blood vessel Rd1 extends specified in the process performed by blood vessel diameter calculating section 12b.

At this time, blood vessel diameter calculating section 12b may calculate, based on the size of tomographic image R1, the actual diameter of blood vessel Rd1 from the diameter of blood vessel Rd1 calculated as a pixel interval in tomographic image R1.

Blood vessel diameter calculating section 12b is capable of, for example, calculating the diameter of blood vessel Rd1 in each of frames of tomographic image R1 continuously generated by tomographic image generating section 3, and comparing the diameters of blood vessels Rd1 appearing in the individual frames of tomographic image R1.

### Frame-of-Interest Specifying Section 12c

Frame-of-interest specifying section 12c obtains a calculation result of the diameter of blood vessel Rd1 from blood vessel diameter calculating section 12b, and specifies, based on the diameter of blood vessel Rd1 calculated in each of a plurality of frames obtained within a predetermined period of tomographic image R1, a frame of interest corresponding to a frame obtained when the diameter of blood vessel Rd1 is maximum and/or minimum (hereinafter simply referred to as “frame of interest”) from among the plurality of frames. That is, frame-of-interest specifying section 12c specifies a frame of tomographic image R1 as a target to be measured by measuring section 12d from among the plurality of frames obtained within the predetermined period.

In general, blood vessel measurement (for example, IMT measurement or FV measurement) is requested to be performed based on a tomographic image of the blood vessel when the diameter of the blood vessel is maximum and/or minimum while the diameter of the blood vessel periodically changes within one heartbeat, as described above.

For example, a tomographic image of a blood vessel obtained when the blood vessel diameter is minimum is used in IMT measurement, whereas a tomographic image of a blood vessel when the blood vessel diameter is maximum is used in FV measurement. In view of the request, frame-of-interest specifying section 12c automatically extracts a frame of interest including tomographic image R1 captured when the diameter of blood vessel Rd1 is maximum or minimum from among a plurality of frames obtained within a predetermined period.

Preferably, for example, frame-of-interest specifying section 12c functions in response to a measurement start instruction being input by a user (for example, an input operation on icon T3a for an FV measurement start instruction or icon T3b for an IMT measurement start instruction in FIG. 3), and specifies a frame of interest by using, as a population of a search target, a plurality of frames obtained within a predetermined period before a time point at which the measurement start instruction is input, in a group of time-series frames continuously generated by tomographic image generating section 3 (i.e., a group of frames stored in cine memory 13). The predetermined period in this case may be set as a time or the number of frames.

In general, in diagnosis using an ultrasound diagnostic apparatus, a user performs observation in real time while capturing a tomographic image with an ultrasound probe placed on a subject, performs an image storage operation or a freeze operation when a blood vessel region that may be necessary for diagnosis appears and the state of the blood vessel is suitable for blood vessel measurement, and performs detailed observation while holding a tomographic image obtained at the time on a screen. By using, as a population of a search target in frame-of-interest specifying section 12c, a plurality of frames obtained within a predetermined period starting from a timing at which a measurement start instruction is input by a user, frame-of-interest specifying section 12c is capable of specifying a frame of interest in which a tomographic image desired by the user is obtained.

In this case, preferably, frame-of-interest specifying section 12c determines a periodic change in the diameter of blood vessel Rd1 associated with a heartbeat on the basis of the diameter of blood vessel Rd1 calculated in each of time-series frames of tomographic image R1 stored in cine memory 13, and specifies a frame of interest including tomographic image R1 captured when the diameter of blood vessel Rd1 is maximum or minimum from among a plurality of frames obtained within a period during which a periodic change corresponding to one heartbeat occurs. This makes it possible avoid wrong selection of a frame of interest resulting from noise (for example, the diameter of blood vessel Rd1 calculated in tomographic image R1 when the imaging state is unstable).

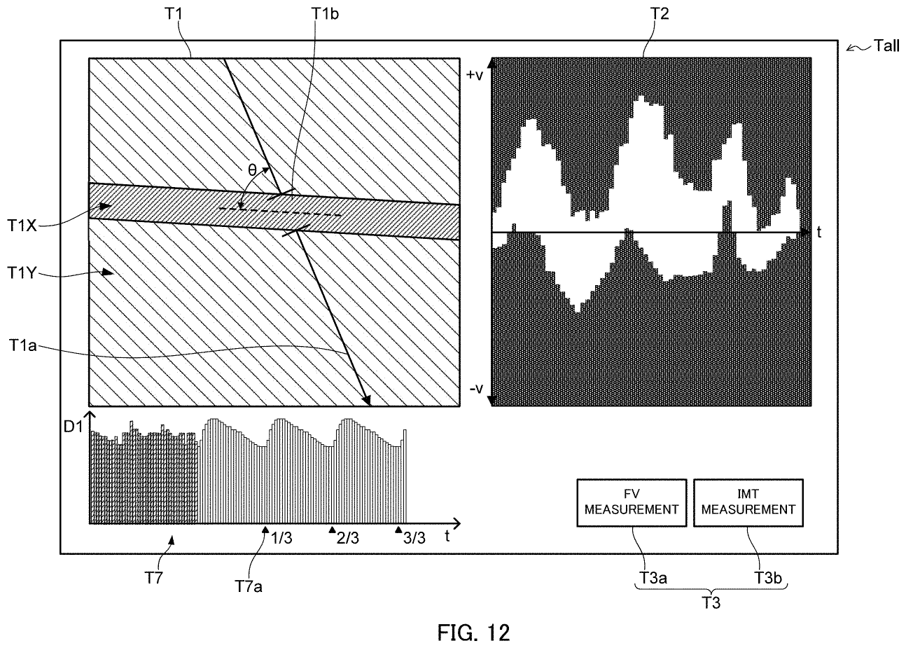

A method for determining a periodic change in the diameter of blood vessel Rd1 by frame-of-interest specifying section 12c may be, for example, autocorrelation computation or frequency analysis. If it is detected that, after a temporal change in the diameter of blood vessel Rd1, a similar periodic change has occurred about three consecutive times, for example, frame-of-interest specifying section 12c may determine the periodic change to be a periodic change in the diameter of blood vessel Rd1 caused by a heartbeat (see, for example, FIG. 12 for details of a periodic change in the diameter of blood vessel Rd1).

Preferably, for example, in response to specifying a frame of interest, frame-of-interest specifying section 12c provides a display instruction to display processing section 5 so as to enable a user to recognize tomographic image R1 as a target of blood vessel measurement, thereby changing the screen displayed on display section 6 from the scanning-operation display screen for displaying a tomographic image continuously generated as a moving image (see FIG. 3) to the measurement display screen for displaying a still image of the frame of interest (see FIG. 4). That is, preferably, display processing section 5 displays a frame of interest of tomographic image R1 as a target of blood vessel measurement in response to the frame of interest being specified by frame-of-interest specifying section 12c.

Such a display process enables the user to determine, before measurement by measuring section 12d is performed (or before measurement by measuring section 12d is completed), whether the frame of tomographic image R1 specified by frame-of-interest specifying section 12c is suitable as a frame of tomographic image as a target of blood vessel measurement. If the user determines that the frame of tomographic image R1 specified by frame-of-interest specifying section 12c is unsuitable as a frame of tomographic image as a target of blood vessel measurement, the user is able to suspend the measurement by measuring section 12d or reset a frame of tomographic image as a target of blood vessel measurement (for example, by using a candidate frame selection function of a fourth modification). Accordingly, the user is able to perform blood vessel measurement by using an appropriate tomographic image while saving time and effort.

### Measuring Section 12d

Measuring section 12d performs measurement of a blood flow of a subject or measurement of the property of a blood vessel of the subject, on blood vessel Rd1 appearing in the frame of interest specified by frame-of-interest specifying section 12c (i.e., the frame obtained when the diameter of blood vessel Rd1 is maximum and/or minimum) of tomographic image R1.

The measurement performed by measuring section 12d may be, for example, FV measurement in blood vessel Rd1 or IMT measurement in blood vessel Rd1, as described above. Measuring section 12d may perform, for example, measurement of the type designated by an input operation performed by a user (for example, an input operation on icon T3a for an FV measurement start instruction or icon T3b for an IMT measurement start instruction in FIG. 3).

For example, when icon T3a for an FV measurement start instruction is selected by the user, measuring section 12d measures an intravascular flow volume (FV) by using the following equation (2) and tomographic image R1 obtained when the diameter of blood vessel Rd1 is maximum.

FV[mL/min]=average blood flow velocity [cm/sec]×blood vessel cross-sectional area [cm²]×60 [sec] Equation (2)

In equation (2), the blood vessel cross-sectional area is calculated by using the diameter of blood vessel Rd1 calculated by blood vessel diameter calculating section 12b, for example, under the assumption that the blood vessel cross section is substantially circular-shaped. As the average blood flow velocity, for example, an average value of the blood flow velocity in blood vessel Rd1 calculated by using equation (1) from the Doppler shift frequency detected by Doppler processing section 4 is used. The average blood flow velocity referred to at this time need not necessarily be an average blood flow velocity at the position of blood vessel Rd1 detected by blood vessel detecting section 12a, and may be an average blood flow velocity of the blood flowing through the same blood vessel as blood vessel Rd1 detected by blood vessel detecting section 12a. In other words, an average blood flow velocity observed at an appropriate timing near blood vessel Rd1 detected by blood vessel detecting section 12a may be used as the average blood flow velocity applied to equation (2).

For example, when icon T3b for an IMT measurement start instruction is selected by the user, measuring section 12d measures an intima-media thickness (IMT) by using tomographic image R1 obtained when the diameter of blood vessel Rd1 is minimum.

FIG. 9 is a diagram for describing an IMT measurement process performed by measuring section 12d. FIG. 9 illustrates an example of an enlarged image of blood vessel Rd1 appearing in tomographic image R1.

A wall portion of blood vessel T1X (an arterial wall herein) typically has a three-layer structure including intima T1X_a1, media T1X_a2, and adventitia T1X_a3. The IMT is the sum of the thickness of intima T1X_a1 and the thickness of media T1X_a2 (i.e., the distance from the boundary between an intravascular lumen and intima T1X_a1 to the boundary between media T1X_a2 and adventitia T1X_a3, width D2 in FIG. 9).

Measuring section 12d performs image analysis to detect the boundary between the intravascular lumen and intima T1X_a1 and the boundary between media T1X_a2 and adventitia T1X_a3 in blood vessel Rd1 appearing in tomographic image R1, and measures the distance between the two boundaries (width D2 in FIG. 9), thereby measuring the IMT. The method of the image analysis for detecting the boundaries is not specified. For example, measuring section 12d detects these boundary positions by using a known edge detection method.

With the above-described process, measuring section 12d automatically performs measurement on blood vessel Rd1 detected by blood vessel detecting section 12a without requiring an input operation by a user.

### Operation of Control Device 10

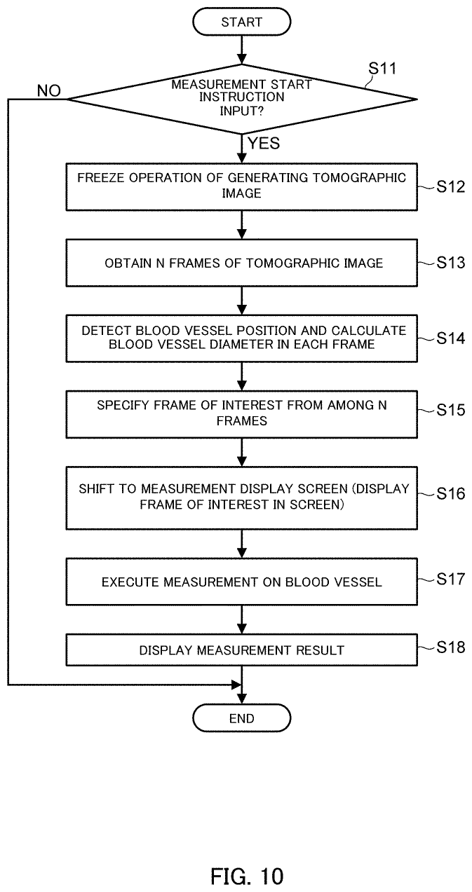

FIG. 10 is a flowchart illustrating an example of operation of control device 10. The flowchart illustrated in FIG. 10 represents steps sequentially executed by control device 10 in accordance with a computer program (i.e., the above-described functions of blood vessel detecting section 12a, blood vessel diameter calculating section 12b, frame-of-interest specifying section 12c, and measuring section 12d).

First, in step S11, control device 10 determines whether a measurement start instruction has been input from a user. If a measurement start instruction has been input from a user (for example, an input operation on icon T3a for an FV measurement start instruction or icon T3b for an IMT measurement start instruction in FIG. 3), the process proceeds to step S12. If a measurement start instruction has not been input from a user, no particular process is performed, and the process of the flowchart in FIG. 10 ends.

In step S12, control device 10 stops an operation of transmitting and receiving ultrasound by ultrasound probe 200, and freezes an operation of generating a tomographic image.

In step S13, control device 10 obtains, as a population of a search target for specifying a frame of interest, N frames of the tomographic image obtained before a time point at which the measurement start instruction is input among time-series frames stored in cine memory 13.

In step S14, control device 10 (blood vessel detecting section 12a and blood vessel diameter calculating section 12b) detects the position of blood vessel Rd1 and calculates the diameter of blood vessel Rd1 in each frame obtained in step S13.

In step S15, control device 10 (frame-of-interest specifying section 12c) specifies a frame of interest corresponding to a frame obtained when the diameter of blood vessel Rd1 is maximum and/or minimum by using data of the diameter of blood vessel Rd1 detected in each of N frames. At this time, for example, control device 10 (frame-of-interest specifying section 12c) determines, based on a temporal change in the diameter of blood vessel Rd1, a periodic change in the diameter of blood vessel Rd1 by autocorrelation computation, and specifies a frame of interest from among a plurality of frames obtained during a periodic change corresponding to one heartbeat.

In step S16, control device 10 (frame-of-interest specifying section 12c) provides a display instruction to display processing section 5 to change the screen displayed on display section 6 from the scanning-operation display screen (see FIG. 3) to the measurement display screen (see FIG. 4).

In step S17, control device 10 (measuring section 12d) executes measurement (for example, IMT measurement or FV measurement) on the blood vessel by using the frame of interest specified in step S15.

In step S18, control device 10 (measuring section 12d) provides a display instruction to display processing section 5 to display, on the measurement display screen (see FIG. 4), the measurement result obtained in step S17.

With the above-described series of steps, control device 10 automatically performs a process of specifying a frame of interest suitable as a measurement target from among frames obtained by a scanning operation during a predetermined period, and executing measurement of the blood flow of the subject or the property of the blood flow of the subject (for example, FV measurement or IMT measurement) by using the frame of interest.

### Advantages

As described above, ultrasound diagnostic apparatus A according to the present embodiment includes:

blood vessel detecting section 12a that detects a region of a blood vessel appearing in a tomographic image;

blood vessel diameter calculating section 12b that calculates a diameter of the blood vessel by analyzing the tomographic image;

frame-of-interest specifying section 12c that specifies, based on the diameter of the blood vessel calculated in each of a plurality of frames obtained within a predetermined period of the tomographic image, a frame of interest from among the plurality of frames, the frame of interest corresponding to a frame obtained when the diameter of the blood vessel is maximum and/or minimum;

measuring section 12d that performs measurement on the blood vessel appearing in the frame of interest of the tomographic image, the measurement being measurement of a blood flow of a subject or a property of the blood vessel of the subject; and

display processing section 5 that displays the frame of interest of the tomographic image at a predetermined timing.

Thus, with ultrasound diagnostic apparatus A according to the present embodiment, it is possible to automatically specify a tomographic image related to a frame of interest obtained when the diameter of a blood vessel is maximum and/or minimum from among a plurality of frames obtained within a predetermined period (for example, within a period of one heartbeat) of the tomographic image, and perform blood vessel measurement.

This enables a user to omit a complicated operation of searching a group of frames stored in a cine memory for a tomographic image suitable for blood vessel measurement. In addition, this makes it possible to accurately select a tomographic image related to a frame of interest obtained when the diameter of the blood vessel is maximum and/or minimum, and thus the accuracy and reliability of blood vessel measurement can be increased.

### First Modification

In general, in diagnosis using an ultrasound diagnostic apparatus, a user needs to perform an operation (for example, a freeze operation or an image storage operation) on the ultrasound diagnostic apparatus while capturing a tomographic image with an ultrasound probe placed on a subject. Thus, when the user performs an operation, the operation may cause a displacement of the ultrasound probe, and the imaging position of the ultrasound probe may deviate from a position suitable for blood vessel measurement in some cases. In addition, the timing to perform an operation by the user may delay with respect to the timing at which the blood vessel appearing in the tomographic image is in a state suitable for blood vessel measurement.

From the above point of view, in ultrasound diagnostic apparatus A according to the present modification, frame-of-interest specifying section 12c automatically specifies a frame of tomographic image R1 to be used for blood vessel measurement at an appropriate timing at which the blood vessel appearing in the tomographic image is in a state suitable for blood vessel measurement (i.e., the timing at which the imaging state of blood vessel Rd1 becomes stable). That is, frame-of-interest specifying section 12c according to the present modification specifies a frame of interest in response to detection that the imaging state of blood vessel Rd1 has become stable in analysis of tomographic image R1, instead of a measurement start instruction from a user.

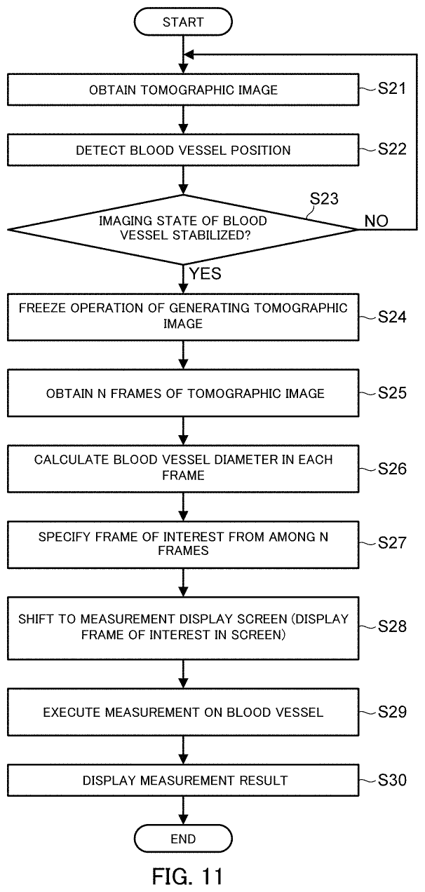

FIG. 11 is a flowchart illustrating an example of operation of control device 10 of ultrasound diagnostic apparatus A according to the present modification. The flowchart in FIG. 11 is different from the flowchart in FIG. 10 in that steps S21 to S23 for determining the stability of the imaging state of blood vessel Rd1 are performed before a process of specifying a frame of interest.

Specifically, in ultrasound diagnostic apparatus A according to the present modification, frame-of-interest specifying section 12c monitors tomographic image R1 updated in real time by tomographic image generating section 3, and determines the sharpness of the image of blood vessel Rd1 detected in tomographic image R1 by blood vessel detecting section 12a. When a state in which the sharpness of the image of blood vessel Rd1 is higher than or equal to a threshold continues for a predetermined period, for example, frame-of-interest specifying section 12c outputs a determination result indicating that the imaging state of blood vessel Rd1 has become stable (steps S21 to S23).

The sharpness of the image of blood vessel Rd1 can be defined as, for example, the sharpness of the outline of a blood vessel wall in the image of blood vessel Rd1. Frame-of-interest specifying section 12c is capable of, for example, calculating an edge detection value (for example, a secondary differential value) of the outline of the wall of blood vessel Rd1 by filtering process, and using the value obtained thereby as the sharpness of the image of blood vessel Rd1.

The process of determining the stability of the imaging state of blood vessel Rd1 by frame-of-interest specifying section 12c may be performed using another method. For example, frame-of-interest specifying section 12c may determine whether the imaging state of blood vessel Rd1 has become stable, in response to detecting a periodic change in the diameter of blood vessel Rd1 associated with heartbeats from a temporal change in the diameter of blood vessel Rd1.

Frame-of-interest specifying section 12c according to the present modification specifies a frame of interest in which the diameter of blood vessel Rd1 is maximum and/or minimum by using, as a population of a search target, a plurality of frames obtained within a predetermined period that is before a time point at which stabilization of the imaging state of blood vessel Rd1 is detected among time-series frames stored in cine memory 13.

Steps S24 to S30 in the flowchart in FIG. 11 correspond to steps S12 to S18 in the flowchart in FIG. 10, respectively, and thus the description thereof is omitted here.

As described above, in ultrasound diagnostic apparatus A according to the present modification, frame-of-interest specifying section 12c specifies a frame of interest in response to detecting that the imaging state of blood vessel Rd1 has become stable. Thus, it is possible to accurately specify tomographic image R1 to be used for blood vessel measurement without requiring a user operation.

### Second Modification

In general, the diameter of a blood vessel periodically changes within one heartbeat. When the imaging state of a tomographic image is instable, it may be difficult for a user to accurately grasp the periodic change if the user merely views the tomographic image updated in real time (i.e., a moving image). In such a state, the diameter of blood vessel Rd1 output from blood vessel diameter calculating section 12b includes noise data, and thus frame-of-interest specifying section 12c may fail in selecting a frame of a tomographic image obtained when the diameter of blood vessel Rd1 is maximum and/or minimum.

From the above point of view, ultrasound diagnostic apparatus A according to the present modification (display processing section 5) displays, on display section 6, a temporal change in the diameter of blood vessel Rd1 calculated in each of time-series frames of tomographic image R1 that is continuously generated.

FIG. 12 is a diagram illustrating an example of image T7 showing a temporal change in the diameter of blood vessel Rd1 (hereinafter referred to as “blood vessel diameter transition image T7”) displayed by ultrasound diagnostic apparatus A according to the present modification. FIG. 12 illustrates a state in which blood vessel diameter transition image T7 is displayed in a display image generated when a scanning operation is executed.

Specifically, in ultrasound diagnostic apparatus A according to the present modification, blood vessel detecting section 12a monitors tomographic image R1 that is updated in real time by tomographic image generating section 3 and detects blood vessel Rd1 in tomographic image R1, and blood vessel diameter calculating section 12b calculates the diameter of blood vessel Rd1. Blood vessel diameter calculating section 12b provides data of the diameter of blood vessel Rd1 to display processing section 5, and display processing section 5 generates a display image regarding the temporal change in the diameter of blood vessel Rd1.

In blood vessel diameter transition image T7 in FIG. 12, the horizontal axis corresponds to a time axis, and the temporal change in the diameter of blood vessel Rd1 is expressed by graphic display in which the diameter of blood vessel Rd1 detected in tomographic image R1 at each timing is represented by the height of a bar in a graph.

In blood vessel diameter transition image T7 in FIG. 12, when a periodic change in the diameter of blood vessel Rd1 starts to be observed, the color of the bars representing the diameter of blood vessel Rd1 in that time period (for example, blue) is made different from the color of the bars representing the diameter of blood vessel Rd1 in the time period in which no periodic change in the diameter of blood vessel Rd1 is observed (for example, red) so that the user is able to easily recognize that the imaging state of the tomographic image has become stable.

In blood vessel diameter transition image T7 in FIG. 12, image T7a (represented by a black triangle mark in FIG. 12) indicating the starting point of a periodic change in the diameter of blood vessel Rd1 (i.e., the timing at which the diameter of blood vessel Rd1 becomes maximum or minimum) is added so that the user is able to easily recognize the periodic change in the diameter of blood vessel Rd1. In FIG. 12, image T7a has information indicating the number of times (“1/3”, “2/3”, and “3/3”) from when the periodic change in the diameter of blood vessel Rd1 starts to be observed.

As described above, ultrasound diagnostic apparatus A according to the present modification provides blood vessel diameter transition image T7 to a user, thereby being able to assist the user in determining the stability of the imaging state of a tomographic image and in recognizing the behavior of the periodic change in the diameter of blood vessel Rd1.

### Third Modification

In general, an ultrasound diagnostic apparatus has a function of displaying a cine bar serving as a user interface in a display screen so that a user is able to view a desired frame among time-series frames stored in a cine memory by execution of a scanning operation.

In this function, it may be convenient for the user if a tomographic image obtained when the diameter of blood vessel Rd1 is maximum and/or minimum is easily selectable. This is because, as described above, frame-of-interest specifying section 12c may fail in determining a frame of a tomographic image obtained when the diameter of blood vessel Rd1 is maximum and/or minimum when the imaging state of the tomographic image is instable, whereas the selection function enables the user to easily determine whether the frame of interest specified by frame-of-interest specifying section 12c is appropriate as a frame of a tomographic image which is a target of blood vessel measurement.

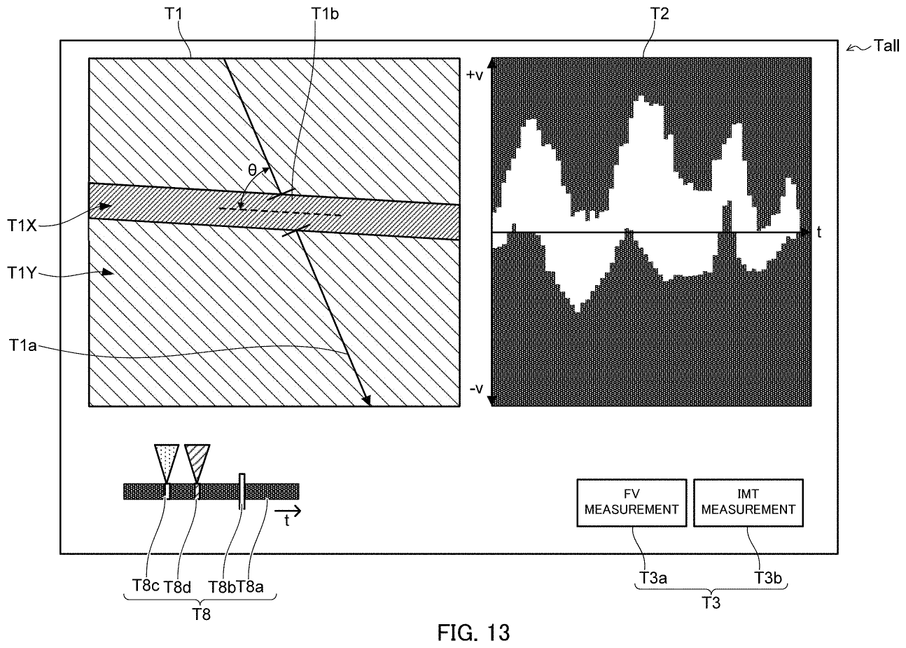

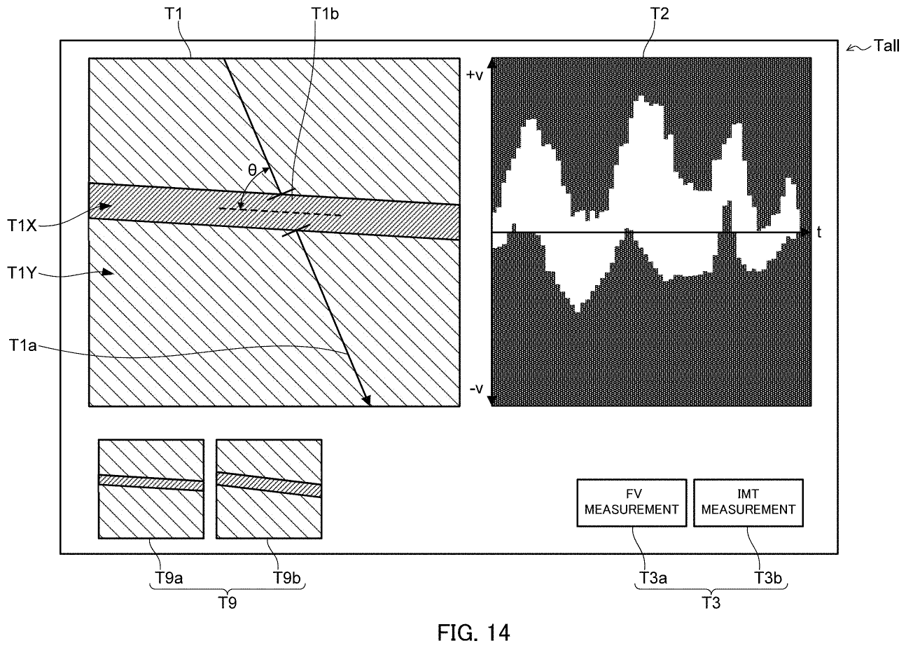

FIG. 13 is a diagram illustrating an example of display of a cine bar in ultrasound diagnostic apparatus A according to the present modification. FIG. 13 illustrates a state in which cine bar T8 is displayed in a display image generated when a scanning operation is executed.

In this example, a user is able to select, by operating cine bar T8, a frame of a tomographic image to be displayed from among time-series frames stored in cine memory 13 by executing a scanning operation, for example, when temporarily freezing the scanning operation. In FIG. 13, the frame of the tomographic image selected as a target to be displayed by an operation of cine bar T8 is displayed in region T1 in display image Tall.

Cine bar T8 is constituted by, for example, bar body T8a and operation knob T8b. Bar body T8a extends in a right-left direction, and the individual frames stored in cine memory 13 are associated therewith in time series in the right-left direction. Operation knob T8b is an operation element capable of moving on bar body T8a in the right-left direction. In response to operation knob T8b being operated to move on cine bar T8, one frame corresponding to the position of operation knob T8b is selected from among the frames stored in cine memory 13, and the tomographic image corresponding to the selected frame is displayed in region T1 in display image Tall.

Cine bar T8 according to the present modification has markers indicating the positions of frames of interest specified by frame-of-interest specifying section 12c (position T8c of the tomographic image obtained when the diameter of blood vessel Rd1 is maximum, and position T8d of the tomographic image obtained when the diameter of blood vessel Rd1 is minimum).

As described above, in ultrasound diagnostic apparatus A according to the present modification, a user is able to easily check, by operating cine bar T8, the sharpness and so forth of the tomographic image of the frame of interest specified by frame-of-interest specifying section 12c of the tomographic image.

### Fourth Modification

In general, the diameter of a blood vessel periodically changes within one heartbeat. Thus, depending on the period of a search target in cine memory 13 captured by frame-of-interest specifying section 12c, there may be a plurality of frames in which the diameter of blood vessel Rd1 is maximum or minimum (hereinafter referred to as “candidate frames of a frame of interest”). In such a case, it is convenient if a user is able to select a frame of interest from among a plurality of candidate frames in consideration of the sharpness or the like of each candidate frame of the tomographic image, instead of automatically specifying a frame of interest in accordance with only the value of the diameter of blood vessel Rd1.

From the above point of view, in a case where a plurality of frames obtained within a predetermined period include two or more candidate frames of a frame of interest in which the diameter of blood vessel Rd1 is maximum or minimum, ultrasound diagnostic apparatus A (frame-of-interest specifying section 12c) according to the present modification is capable of causing display processing section 5 to display each of the two or more candidate frames, and selecting one of the two or more candidate frames as a frame of interest by a user operation.

FIG. 14 is a diagram illustrating an example of candidate frame display (hereinafter referred to as “frame-of-interest selection image T9”) displayed by ultrasound diagnostic apparatus A according to the present modification. FIG. 14 illustrates a state in which frame-of-interest selection image T9 is displayed in a display image generated when a scanning operation is executed.

Frame-of-interest selection image T9 in FIG. 14 is an example of a display manner in a case where a plurality of frames obtained within a predetermined period include two candidate frames in which the diameter of blood vessel Rd1 is maximum. Two candidate frames T9a and T9b are displayed in frame-of-interest selection image T9 in FIG. 14. A user is able to select either candidate frame T9a or candidate frame T9b as a frame of interest by a selection operation using operation input section 7.

As described above, in ultrasound diagnostic apparatus A according to the present modification, a user is able to select a tomographic image to be used for blood vessel measurement, and it is possible to meet more various demands of the user.

The frame-of-interest manual selection function according to the present modification may be implemented, for example, when the user has input an operation for interrupting measurement by measuring section 12d or resetting a tomographic image as a target of blood vessel measurement (for example, using the candidate frame selection function of the fourth modification). In addition, it may be possible to set which of the frame-of-interest manual selection function according to the present modification and the frame-of-interest automatic selection function of frame-of-interest specifying section 12c is to be used.

### Fifth Modification

In general, which of a tomographic image obtained when the diameter of a blood vessel is maximum and a tomographic image obtained when the diameter of a blood vessel is minimum in a periodic change within one heartbeat is to be used as a tomographic image for blood vessel measurement depends on a diagnosis method using a measurement result of each user.

From the above point of view, ultrasound diagnostic apparatus A (control device 10) according to the present modification includes a setting section (not illustrated) that enables a user setting to be made regarding which of a maximum-blood-vessel frame obtained when the diameter of blood vessel Rd1 is maximum, a minimum-blood-vessel frame obtained when the diameter of blood vessel Rd1 is minimum, and a set of the maximum-blood-vessel frame and the minimum-blood-vessel frame among a plurality of frames is regarded as a frame of interest which is a target to be specified by frame-of-interest specifying section 12c.

Preferably, the setting section makes the target to be specified as a frame of interest settable to frame-of-interest specifying section 12c for each of types of blood vessel measurement using a tomographic image or each of types of imaging target of a tomographic image.

The setting section causes display processing section 5 to display a user interface image that enables these items to be set.

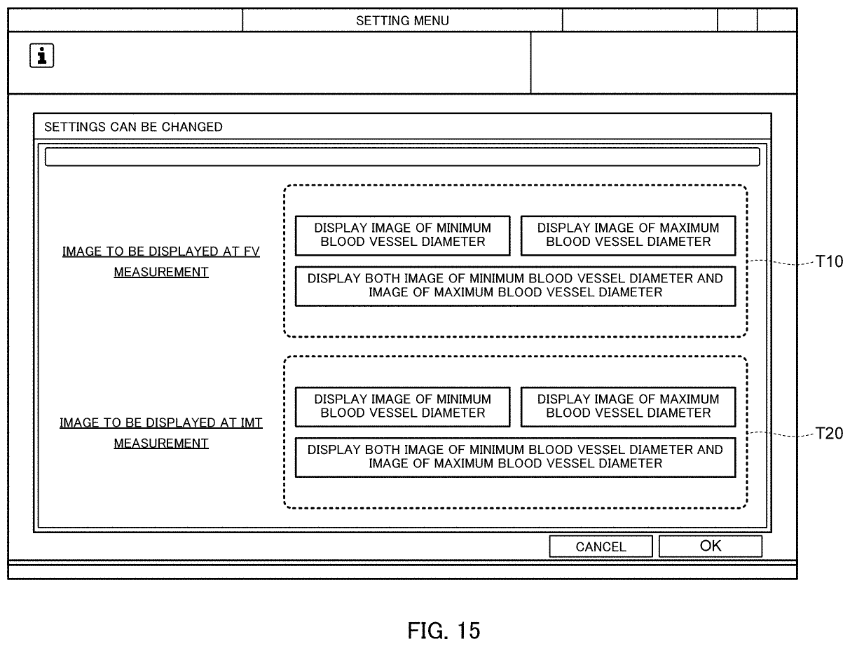

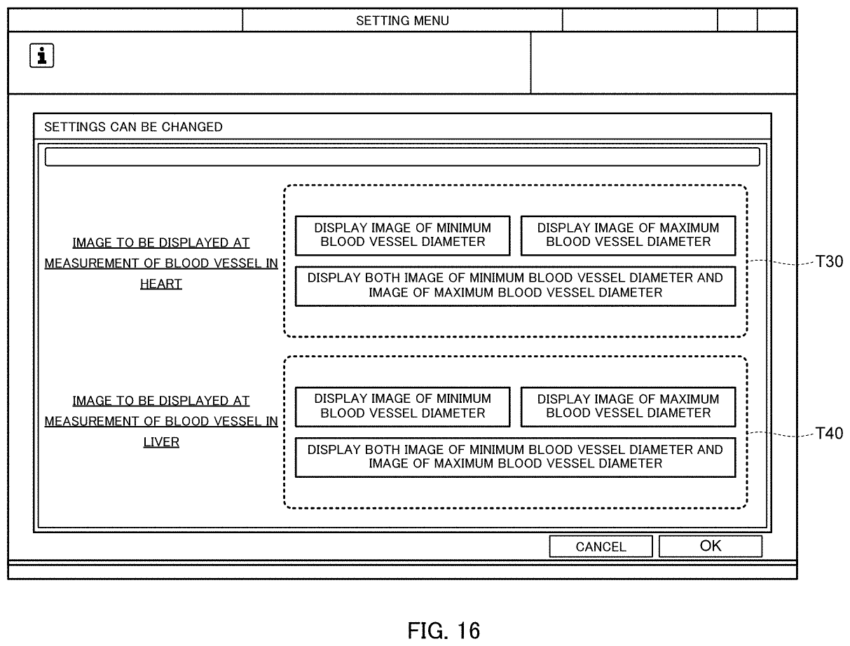

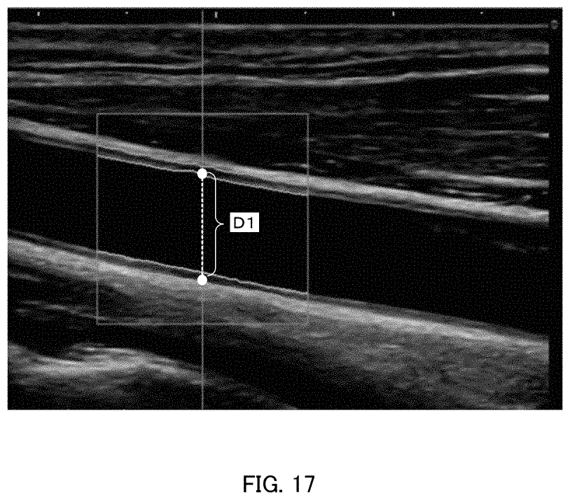

FIGS. 15 and 16 are diagrams each illustrating an example of a user interface image that is caused to be displayed by the setting section of ultrasound diagnostic apparatus A according to the present modification.

FIG. 15 illustrates a state in which icon groups T10 and T20 are displayed so as to enable a setting to be made regarding which of a maximum-blood-vessel frame, a minimum-blood-vessel frame, and a set of the maximum-blood-vessel frame and the minimum-blood-vessel frame is regarded as a frame of interest which is a target to be specified by frame-of-interest specifying section 12c for each of types of blood vessel measurement (FV measurement and IMT measurement herein).

FIG. 16 illustrates a state in which icon groups T30 and T40 are displayed so as to enable a setting to be made regarding which of a maximum-blood-vessel frame, a minimum-blood-vessel frame, and a set of the maximum-blood-vessel frame and the minimum-blood-vessel frame is regarded as a frame of interest which is a target to be specified by frame-of-interest specifying section 12c for each of types of imaging target of a tomographic image (measurement of a blood vessel in the heart and measurement of a blood vessel in the liver herein).

As described above, ultrasound diagnostic apparatus A according to the present modification is capable of setting a target desired by a user as a tomographic image used for blood vessel measurement, and meeting more various demands of the user.

### Other Embodiments

The present disclosure is not limited to the above embodiment and is applicable to various modifications.

For example, in the above embodiment, a blood vessel longer-axis image is illustrated as an image of a blood vessel to be detected in tomographic image R1 by blood vessel detecting section 12a. However, as an image of a blood vessel to be detected in tomographic image R1 by blood vessel detecting section 12a, a blood vessel shorter-axis image, which is a cross section in a shorter-length direction of a blood vessel, may be used together with or instead of a blood vessel longer-axis image.

In this case, for example, blood vessel detecting section 12a may detect blood vessel Rd1 in tomographic image R1 by using template image Rw of a blood vessel shorter-axis image together with or instead of a blood vessel longer-axis image. In a case where blood vessel Rd1 appearing in tomographic image R1 is a shorter-axis image or in a case where it is not known whether blood vessel Rd1 appearing in tomographic image R1 is a longer-axis image or a shorter-axis image, a known method described in, for example, Japanese Patent Application Laid-Open No. 2008-253379 may be used to calculate the diameter of blood vessel Rd1.

In the above embodiment, frame-of-interest specifying section 12c specifies a frame of interest and causes display section 6 to display the frame of interest of the tomographic image when a measurement start instruction is input by a user or when a determination is made that the imaging state of a tomographic image has become stable. However, frame-of-interest specifying section 12c may specify a frame of interest and cause display section 6 to display the frame of interest of the tomographic image at another timing, for example, when a freeze operation is input by a user or when a B-mode operation is suspended and a PW-Doppler-mode operation is activated. In this case, it is preferable that measurement by measuring section 12d be performed in response to input of a measurement start instruction by the user.

Various modifications can also be made as for the manner of displaying a frame of interest of tomographic image R1 as a target of blood vessel measurement by display processing section 5.

For example, display processing section 5 may display enlarged tomographic image R1 at the time of starting blood vessel measurement such as FV measurement. When the size of a blood vessel depicted in tomographic image R1 is small, it is difficult to determine whether a candidate point of a blood vessel measurement point is appropriate and to correct the point. The determination and correction is facilitated by displaying enlarged tomographic image R1. In other words, if tomographic image R1 is constantly displayed in an enlarged manner when blood vessel measurement is not being performed, it may be difficult to determine the state around the enlarged area, and the visibility of tomographic image R1 may decrease. Thus, it is preferable to display tomographic image R1 in an enlarged manner only at the time of starting blood vessel measurement. Whether enlargement is to be performed may be made settable at the time of selecting a frame or starting blood vessel measurement.

Display processing section 5 may display tomographic image R1 in the entire display screen by zooming it in at the timing of selecting a frame of interest in which the blood vessel diameter is maximum or minimum.

Although embodiments of the present invention have been described and illustrated in detail, the disclosed embodiments are made for purpose of illustration and example only and not limitation. The scope of the present invention should be interpreted by terms of the appended claims.

What is claimed is:

1. An ultrasound diagnostic apparatus that generates a tomographic image of a subject by transmitting and receiving ultrasound, the ultrasound diagnostic apparatus comprising:
a hardware processor that:
detects a region of a blood vessel appearing in the tomographic image;
calculates a diameter of the blood vessel by analyzing the tomographic image;
specifies, based on the diameter of the blood vessel calculated in each of a plurality of frames obtained within a predetermined period of the tomographic image, a frame of interest from among the plurality of frames, the frame of interest corresponding to a frame obtained when the diameter of the blood vessel is maximum and/or minimum;
performs measurement on the blood vessel appearing in the frame of interest of the tomographic image, the measurement being measurement of a blood flow of the subject or a property of the blood vessel of the subject; and
displays the frame of interest of the tomographic image at a predetermined timing.

2. The ultrasound diagnostic apparatus according to claim 1, wherein in response to a measurement start instruction for performing the measurement being input by a user, the hardware processor specifies the frame of interest from among the plurality of frames obtained within the predetermined period that is before a time point at which the measurement start instruction is input.

3. The ultrasound diagnostic apparatus according to claim 1, wherein in response to detecting that an imaging state of the blood vessel has become stable, the hardware processor specifies the frame of interest from among the plurality of frames obtained within the predetermined period that is before a time point at which the imaging state of the blood vessel becomes stable.

4. The ultrasound diagnostic apparatus according to claim 1, wherein in response to specifying the frame of interest, the hardware processor displays the frame of interest of the tomographic image.

5. The ultrasound diagnostic apparatus according to claim 1, wherein in response to specifying the frame of interest, the hardware processor causes a shift from a first screen mode of displaying a moving image of the tomographic image that is continuously generated to a second screen mode of displaying a still image of the frame of interest of the tomographic image.

6. The ultrasound diagnostic apparatus according to claim 1, wherein based on the diameter of the blood vessel calculated in each of time-series frames of the tomographic image that is continuously generated, the hardware processor determines a periodic change in the diameter of the blood vessel associated with heartbeats, and specifies the frame of interest from among the plurality of frames obtained during the periodic change corresponding to one heartbeat.

7. The ultrasound diagnostic apparatus according to claim 1, wherein the hardware processor displays a temporal change in the diameter of the blood vessel calculated in each of time-series frames of the tomographic image that is continuously generated.

8. The ultrasound diagnostic apparatus according to claim 1, wherein in a case where the plurality of frames obtained within the predetermined period include two or more candidate frames of the frame of interest in which the diameter of the blood vessel is maximum or minimum, the hardware processor displays each of the two or more candidate frames such that one of the two or more candidate frames is selectable as the frame of interest by a user operation.

9. The ultrasound diagnostic apparatus according to claim 1, wherein the hardware processor displays, by a user operation, a cine bar that makes a target frame to be displayed selectable from among time-series frames of the tomographic image that is continuously generated, and adds, by a user operation, marker display indicating a position corresponding to the frame of interest to the cine bar.

10. The ultrasound diagnostic apparatus according to claim 1, wherein the hardware processor enables a user setting to be made regarding which of a maximum-blood-vessel frame obtained when the diameter of the blood vessel is maximum, a minimum-blood-vessel frame obtained when the diameter of the blood vessel is minimum, and a set of the maximum-blood-vessel frame and the minimum-blood-vessel frame among the plurality of frames is regarded as the frame of interest which is a target to be specified by the hardware processor.

11. The ultrasound diagnostic apparatus according to claim 10, wherein the hardware processor makes a target to be specified as the frame of interest settable for each of types of the measurement using the tomographic image or each of types of imaging target of the tomographic image.

12. The ultrasound diagnostic apparatus according to claim 1, wherein the measurement is flow volume (FV) measurement in the blood vessel of the subject or intima-media thickness (IMT) measurement in the blood vessel of the subject.

13. The ultrasound diagnostic apparatus according to claim 1, further comprising:
an ultrasound probe that transmits the ultrasound toward the subject and receives a reflected wave echo of the ultrasound from an inside of the subject.

14. A method for controlling an ultrasound diagnostic apparatus that generates a tomographic image of a subject by transmitting and receiving ultrasound, the method comprising:
detecting a region of a blood vessel appearing in the tomographic image;
calculating a diameter of the blood vessel by analyzing the tomographic image;
specifying, based on the diameter of the blood vessel calculated in each of a plurality of frames obtained within a predetermined period of the tomographic image, a frame of interest from among the plurality of frames, the frame of interest corresponding to a frame obtained when the diameter of the blood vessel is maximum and/or minimum;
performing measurement on the blood vessel appearing in the frame of interest of the tomographic image, the measurement being measurement of a blood flow of the subject or a property of the blood vessel of the subject; and
displaying the frame of interest of the tomographic image at a predetermined timing.

15. A non-transitory computer-readable recording medium storing a program for controlling an ultrasound diagnostic apparatus that generates a tomographic image of a subject by transmitting and receiving ultrasound, the program causing a computer to perform:
detecting a region of a blood vessel appearing in the tomographic image;
calculating a diameter of the blood vessel by analyzing the tomographic image;
specifying, based on the diameter of the blood vessel calculated in each of a plurality of frames obtained within a predetermined period of the tomographic image, a frame of interest from among the plurality of frames, the frame of interest corresponding to a frame obtained when the diameter of the blood vessel is maximum and/or minimum;
performing measurement on the blood vessel appearing in the frame of interest of the tomographic image, the measurement being measurement of a blood flow of the subject or a property of the blood vessel of the subject; and
displaying the frame of interest of the tomographic image at a predetermined timing.
